(19) United States
(12) Patent Application Publication
(10) Pub. No.: US 2014/0148407 A1
(43) Pub. Date: May 29, 2014

(54) AQUEOUS GELS

(71) Applicants: Janet Lesley Scott, Bath (GB); Christopher Smith, Mansfield (GB); Giovanni Francesco Unali, Bebington (GB)

(72) Inventors: Janet Lesley Scott, Bath (GB); Christopher Smith, Mansfield (GB); Giovanni Francesco Unali, Bebington (GB)

(21) Appl. No.: 14/124,074

(22) PCT Filed: May 9, 2012
(86) PCT No.: PCT/EP2012/058523

§ 371 Date: Feb. 4, 2014

(30) Foreign Application Priority Data
Jun. 16, 2011 (EP) 11170246.0

(51) Int. Cl. A61K 8/73; A61Q 19/00; A61Q 5/00

(52) U.S. Cl. CPC A61K 8/731; A61Q 5/00; A61Q 19/00
USPC 514/57

(57) ABSTRACT

The invention provides an aqueous gel comprising: a) from 0.5 to 5 wt % dispersed modified cellulose biopolymer, wherein the modification consists of the cellulose having its C6 primary alcohols oxidised to carboxyl moieties (acid/COOH—) on 10 to 70% of the glucose units and substantially all the remainder of the C6 positions occupied by unmodified primary alcohols; b) a water-soluble or water-miscible organic non-solvent for the modified cellulose biopolymer; c) 0 to 10 wt % non-surfactant electrolyte, and d) water; in which the aqueous gel comprises less than 3 wt % oil phase ingredients. The aqueous gels of the invention offer excellent tactile properties, in particular superior skin feel and reduced stickiness. Furthermore, the gels have thixotropic properties, allowing their usage in pumpable or sprayable formats. They also provide sufficient structure for the suspension of a variety of particulate materials.

### BACKGROUND

Cellulose is a plentiful, and consequently inexpensive, biopolymer. However, in its unmodified form it is completely insoluble and cannot be dispersed into an aqueous liquid composition to achieve a stable, thickened, product.

Partially and selectively oxidising cellulose at the C6 position creates cellouronates or cellouronic acids which are more water dispersible than cellulose but still relatively insoluble.

WO 2010/076292 describes how this type of oxidised cellulose may be used as an alternative structurant for aqueous detergent compositions. This enables the formulator to replace surfactant required for structuring with relatively low concentrations of low cost, partially oxidised, dispersed modified cellulose.

We have now found that the oxidised cellulose described in WO2010/076292 can be formulated with water-miscible alcohols or polyols to give aqueous gels with excellent tactile properties, in particular superior skin feel and reduced stickiness. Furthermore, the gels have thixotropic properties, allowing their usage in pumpable or sprayable formats. They also provide sufficient structure for the suspension of a variety of particulate materials.

### SUMMARY OF THE INVENTION

According to a first aspect of the present invention there is provided an aqueous gel comprising:
a) from 0.5 to 5 wt % dispersed modified cellulose biopolymer, wherein the modification consists of the cellulose having its C6 primary alcohols oxidised to carboxyl moieties (acid/COOH—) on 10 to 70% of the glucose units and substantially all the remainder of the C6 positions occupied by unmodified primary alcohols;
b) a water-soluble or water-miscible organic non-solvent for the modified cellulose biopolymer;
c) 0 to 10 wt % non-surfactant electrolyte, and
d) water;
   in which the aqueous gel comprises less than 3 wt % oil phase ingredients.

Also, according to a second aspect of the invention, there is provided a process to manufacture an aqueous gel according to the first aspect, the process comprising the steps of:
(i) dispersing 0.5 to 5 wt % modified cellulose biopolymer in water under high shear to hydrate it, wherein the modification consists of the cellulose having its C6 primary alcohols oxidised to carboxyl moieties (acid/COOH—) on 10 to 70% of the glucose units and substantially all the remainder of the C6 positions occupied by unmodified primary alcohols;
(ii) adding to this aqueous dispersion a water-soluble or water-miscible organic non-solvent for the modified cellulose biopolymer, and
(iii) optionally also adding up to 10 wt % non-surfactant electrolyte.

### DETAILED DESCRIPTION OF THE INVENTION

### Modified Cellulose Biopolymer

The aqueous gel of the present invention comprises from 0.5 to 5 wt % dispersed modified cellulose biopolymer, wherein the modification consists of the cellulose having its C6 primary alcohols oxidised to carboxyl moieties (acid/COOH—) on 10 to 70% of the glucose units and substantially all the remainder of the C6 positions occupied by unmodified primary alcohols.

The modified cellulose biopolymer for use in the invention may be characterised as a water insoluble, water dispersible modified cellulose in which only a proportion of its C6 primary alcohol groups have been oxidised to acid groups.

Cellulose where all such alcohols have been oxidised is called polyuronic acid or polyglucuronic acid. Such fully oxidised material is soluble in water. It is unsuitable for use in the present invention for two reasons. Firstly, the cost of the extra processing required to create more than 70% substitution of primary alcohols by carboxylic acid groups makes it not cost effective as a replacement for surfactant and second the highly oxidised material tends to include unwanted depolymerised cellulose, which leads to a reduction of yield of insoluble dispersible structurant.

In the context of the present invention, a modified cellulose biopolymer is said to be water soluble, if it leaves less than 10 wt % of its dry mass as undissolved residue when a 2g dry sample is added to 1 litre of agitated demineralised water at 25° C.

Totally unoxidised (unmodified) cellulose is unable to function as a structurant. Oxidising the cellulose to have at least 10% of the primary alcohols converted to carboxylic acids makes the cellulose dispersible in water and when mixed within the surfactant system the resulting structured liquid or gel maintains the cellulose in a dispersed state so it does not settle over time.

### The Cellulose Starting Material

Several factors influence the choice of a suitable starting material.

More porous unmodified cellulosic material will oxidise more rapidly. Characterisation of surface area or porosity is readily achieved by porosimetry or BET measurements. In general, those starting materials that oxidise more rapidly due to their low crystallinity and higher surface area and/or porosity, prove easier to disperse than those that oxidise less rapidly.

The rate of oxidation is also affected by the dimensions of the particles of cellulose starting material; the reduction in rate for longer (>500 micron) fibres is significant. Fibres less than 500 microns long are therefore preferred for this reason and due to the added difficulty in agitation of the longer fibres. While oxidation results in significant gross particle size reduction, this does not compensate for decreased fibril surface accessibility in the long fibres.

Celluloses that have not been previously subjected to acid hydrolysis are a preferred starting material, due to reactivity, cost and resultant product dispersibility.

Relatively unrefined α-cellulose, for example filter aid fibres, provides one of the most readily oxidised and dispersed sources of cellulose. Advantageously, the oxidation process also serves to bleach coloured components, such as lignin, in such unbleached cellulose starting materials. This then renders such materials more suitable for use in contexts where visual clarity of the end product is desirable, for example transparent personal care formulations.

### Oxidation

Because of its known specificity for primary alcohol oxidation TEMPO-mediated oxidation of cellulose is preferred (i.e. 2,2,6,6-tetramethylpiperidine-1-oxyl and related nitroxy radical species). The process proceeds well without cooling, at relatively high weight % cellulose in the initial suspension. Simple workup procedures afford clean material suitable for dispersion. Such TEMPO mediated oxidation of cellulose is described in the published literature and the skilled worker will be able as a matter of routine to adapt known methods to achieve the oxidation required by this invention.

While aqueous NaOCl/TEMPO/NaBr is a highly preferred oxidation system, there are a number of other systems available to the skilled worker, especially for large scale production. Among such systems, there may be mentioned use of peracetic acid or monoperoxysulfate salts (Oxone®) as the oxidant with 4-acetamido-2,2,6,6-tetramethylpiperidine-1-oxyl (4-acetamido-TEMPO) as the radical transfer catalyst or mediator and sodium bromide co-catalyst for the oxidation. Elimination of chlorine from the oxidation system is environmentally desirable.

The use of 4-acetamido-TEMPO as radical transfer catalyst is also advantageous as, although it has a higher molecular weight than TEMPO, it has significantly lower vapour pressure reducing potential exposure hazards. Many other 4-substituted TEMPO analogues exist, but many, such as 4-hydroxy-TEMPO exhibit poor stability. TEMPO on solid supports or on soluble polymers may be used.

Electrochemical oxidation is a potentially clean means of effecting oxidation of carbohydrate moieties, although mediation by a radical transfer catalyst (such as TEMPO) is still required.

Laccase mediated oxidation, which also requires a radical transfer catalyst (e.g. TEMPO) but replaces the oxidant with an enzyme, may advantageously be used.

Using the TEMPO system the degree of reproducibility of oxidation of cellulose from the same source is good.

### Degree of Oxidation

In the context of the present invention, the term “degree of oxidation” of the modified cellulose means the percentage glucose units oxidised to carboxylic acid as measured by titration with sodium hydroxide. It is assumed that all oxidation takes place at the primary alcohol positions. A reasonable assumption, given that primary alcohol specific oxidation chemistry is employed. Furthermore it is assumed that all oxidation leads to carboxylic acid formation.

Degree of polymerisation (DP) does not seem greatly to influence the performance of the modified cellulose. The key thing is that the modified cellulose must remain insoluble.

During oxidation, there is some degradation of the cellulose allowing release of polymer chains. It is particularly advantageous to keep this to a minimum in order to increase the yield of the modified insoluble cellulose material suitable for structuring applications. We have determined that above 70% oxidisation, the yield is unacceptably low and the processing costs become unacceptably high.

The degree of oxidation of the modified cellulose lies in the range 10 to 70%. As the degree of oxidation increases, the amount of soluble material produced will rise and this reduces the yield of insoluble structuring material, thus the higher degrees of oxidation confer no real structuring benefits. For this reason, it is preferred to restrict the degree of oxidation to 60%, or even 50% and the most preferred modified materials have degrees of oxidation even lower than 40% or sometimes even lower than 30%.

To achieve a high enough dispersibility/solubility for the modified cellulose to act as a structurant it must be oxidised to at least 10%. The exact amount of oxidation required for a minimum effect will vary according to the starting material used. Preferably, it is at least 15% oxidised and most preferably, at least 20% oxidised.

### Dispersal of the Modified Cellulose

At small scale, high energy sonication is the preferred method to give the high shear necessary to achieve the aqueous dispersion of the modified cellulose. However, other techniques are more suitable for large scale applications. These include the use of a high speed and high shear stirrer, or a blender, or a homogeniser. Homogenisation may achieve higher levels of dispersed material than are attainable via sonication.

When degrees of oxidation of less than 10% are used, the partially oxidised cellulose proves too resistant to dispersion to produce a transparent or translucent mixture and higher energy input is required. Provided the lower limit of 10% is exceeded, those modified celluloses with a lesser degree of oxidation appear to provide greater structuring capacity once dispersed. This is attributed to less degradation of the material during oxidation and thus the existence of longer individual dispersed (not dissolved) fibrils. This may be because the structure of the cellulose starting material is partially retained, but the fibrils are rendered dispersible by the introduction of negatively charged functional groups on the surface during oxidation.

Oxidised, dispersed cellulose is a largely insoluble polymer that occurs in the form of well dispersed fibrils rather than isolated solvated polymer chains. The fibrils have a large aspect ratio and are thin enough to provide almost transparent dispersions. Carboxylate groups provide anionic surface charge, which results in a degree of repulsion between fibrils, militating against their reassociation into larger structures. Addition of acid to dispersions of oxidised cellulose results in separation of gelled material while at pH between ca 5-9 fibrils may be maintained in a dispersed form as the COO— salt of an appropriate counterion.

Once the high shear dispersion of the modified cellulose has taken place, the remaining process steps can take place in a conventional stirred tank, at relatively low shear. This allows the formulator to make a stock of aqueous dispersion of the modified cellulose, with further ingredients added as and when necessary to enable easy late-stage variations in composition before products are packaged.

The amount of modified cellulose biopolymer in the aqueous gel of the invention preferably ranges from 1 to 2 wt % (by total weight modified cellulose biopolymer based on the total weight of the aqueous gel).

### Water

The amount of water in the aqueous gel of the invention generally ranges from 50 to 95 wt % , and preferably ranges from 70 to 95 wt % (by weight water based on the total weight of the aqueous gel).

### Water-Soluble or Water-Miscible Organic Non-Solvent

The aqueous gel of the present invention comprises a water-soluble or water-miscible organic non-solvent for the modified cellulose biopolymer.

Preferred materials of this class include water-soluble or water-miscible mono- or polyhydric alcohols.

Specific examples of such materials include C₂₋₄ monohydric alcohols, such as ethanol, 1-propanol, 2-propanol (isopropyl alcohol) and tert-butyl alcohol; as well as diols and polyols, such as ethylene glycol, 1,2-propylene glycol, 1,3-butylene glycol, hexylene glycol, diethylene glycol, dipropylene glycol, 2-ethoxyethanol, diethylene glycol monomethyl ether, triethylene glycol monomethyl ether, glycerol (glycerine) and sorbitol.

The most preferred materials are ethanol, glycerol and mixtures thereof.

Mixtures of any of the above described materials may also be used.

The amount of water-soluble or water-miscible organic non-solvent in the aqueous gel of the invention depends on the particular material used, but generally ranges from 0.5 to 50 wt %, and preferably ranges from 1 to 40 wt % (by total weight water-soluble or water-miscible organic non-solvent based on the total weight of the aqueous gel).

### Surfactant

In the aqueous gel of the invention, it is not necessary to use any surfactants in order to provide gelled material. This may be advantageous, for example in formulations designed for topical skin application, or other contexts where skin mildness is particularly desirable.

Accordingly, a preferred aqueous gel according to the invention comprises less than 0.2 wt % anionic surfactant (by total weight anionic surfactant based on the total weight of the aqueous gel).

Examples of anionic surfactants are the sodium, magnesium, ammonium or ethanolamine salts of C₈ to C₁₈ alkyl sulphates (for example sodium dodecyl sulphate), C₈ to C₁₈ alkyl sulphosuccinates (for example dioctyl sodium sulphosuccinate), C₈ to C₁₈ alkyl sulphoacetates (such as sodium dodecyl sulphoacetate), C₈ to C₁₈ alkyl sarcosinates (such as sodium dodecyl sarcosinate), C₈ to C₁₈ alkyl phosphates (which can optionally comprise up to 10 ethylene oxide and/or propylene oxide units) and sulphated monoglycerides.

More preferably the aqueous gel of the invention comprises less than 0.2 wt % total surfactant selected from anionic, amphoteric, cationic and nonionic surfactants respectively. By this is meant that the total content of the anionic surfactant and the amphoteric surfactant and the cationic surfactant and the nonionic surfactant in the aqueous gel of the invention preferably amounts to less than 0.2% by weight based on the total weight of the aqueous gel.

Examples of amphoteric surfactants are alkyl amine oxides, alkyl betaines, alkyl amidopropyl betaines, alkyl sulphobetaines (sultaines), alkyl glycinates, alkyl carboxyglycinates, alkyl amphoacetates, alkyl amphopropionates, alkylamphoglycinates, alkyl amidopropyl hydroxysultaines, acyl taurates and acyl glutamates, wherein the alkyl and acyl groups have from 8 to 19 carbon atoms.

Examples of cationic surfactants are quaternary ammonium salts corresponding to the following general formula (I):

[N (R¹)(R²)(R³)(R⁴)]⁺ (X)⁻ (I)

in which R¹ is an aliphatic group of from 8 to 22 carbon atoms; R², R³, and R⁴ are each independently selected from (a) an aliphatic group of from 1 to 22 carbon atoms, or (b) an aromatic, alkoxy, polyoxyalkylene, alkylamido, hydroxyalkyl, aryl or alkylaryl group having up to 22 carbon atoms; and X is a salt-forming anion such as those selected from halogen, (e.g. chloride, bromide), acetate, citrate, lactate, glycolate, phosphate nitrate, sulphate, and alkylsulphate radicals.

Examples of nonionic surfactants are polyoxyethylene ethers of fatty alcohols, acids and amides, having from 8 to 20 carbon atoms in the fatty chain and from 4 to about 100 oxyethylene units.

Most preferably the aqueous gel is substantially free of surfactant selected from anionic, amphoteric, cationic and nonionic surfactants respectively. The term “substantially free” in this particular context generally means that the total content of the anionic surfactant and the amphoteric surfactant and the cationic surfactant and the nonionic surfactant in the aqueous gel amounts to less than 0.1%, more preferably less than 0.01%, by weight based on the total weight of the aqueous gel.

### Optional Non-Surfactant Electrolyte

The non-surfactant electrolyte is optional. The preferred non-surfactant electrolyte is a water soluble inorganic or organic salt with a molecular weight of less than 500. The electrolyte preferably has a monovalent cation, however at low (less than 2 wt %) levels salts with divalent cations, such as calcium chloride, may be used.

Sodium chloride is the preferred non-surfactant electrolyte.

The amount of non-surfactant electrolyte (such as sodium chloride) in the aqueous gel of the invention generally ranges from 0 to 5 wt %, and preferably ranges from 0 to 2 wt % (by total weight non-surfactant electrolyte based on the total weight of the aqueous gel).

### Oil Phase

The aqueous gel of the invention comprises less than 3 wt %, preferably less than 1 wt % oil phase ingredients (by total weight oil phase ingredients based on the total weight of the aqueous gel) .

The term “oil phase ingredients” in the context of this invention generally means lipophilic materials with a liquid or semi-solid consistency at 25° C.

Specific examples of oil phase ingredients include:

oily or waxy hydrocarbons of synthetic, animal or mineral origin: such as mineral oil, petrolatum, paraffin oils such as isoparaffin, ceresin, ozokerite, squalane, squalene, microcrystalline wax, polyethylene wax, polybutene, polyisobutene, and hydrogenated polyisobutene;

silicones: such as dimethicone, dimethicone copolyol, stearoxy dimethicone, silicone wax and cyclomethicone;

higher fatty acids having 6 to 50, preferably 10 to 20, carbon atoms in a molecule: such as isostearic acid, oleic acid, hexanoic acid and heptanoic acid;

fatty alkyl or alkenyl esters having 6 to 50, preferably 10 to 50, carbon atoms in a molecule: such as cetyl 2-ethylhexanoate, cetyl palmitate, C₁₂₋₁₅ alkyl benzoate, octyl palmitate, octyl hydroxystearate, octyldodecyl myristate, octyldodecyl oleate, decyl oleate, stearyl heptanoate, diisostearyl malate, isopropyl linoleate, isopropyl myristate, isopropyl isostearate, isopropyl palmitate, isocetyl stearate, myristyl myristate, myristyl lactate and propylene glycol dicaprylate/dicaprate;

aliphatic higher alcohols having 6 to 50, preferably 10 to 20 carbon atoms in a molecule: such as cetyl alcohol, stearyl alcohol, isostearyl alcohol and oleyl alcohol;

oily, fatty or waxy esters of natural (plant or animal) origin: such as apricot kernel oil, avocado oil, sweet almond oil, beeswax, castor oil, cocoa butter, lanolin, candelilla wax, carnauba wax, shea butter, shea oil, cereal germ oils, cottonseed oil, corn oil, jojoba oil, safflower oil, sunflower oil, olive oil, rapeseed oil, soybean oil, palm kernel oil, babassu kernel oil, coconut oil and medium-chain triglyceride (MCT) oils, which may generally be defined as mixtures of medium chain saturated fatty acids ranging from caproic to lauric (C₆ to C₁₂), in their triglyceride form, and which are typically obtainable from the fractionation of coconut oil.

### Product Applications, Formulations and Optional Ingredients

The aqueous gels of the present invention may advantageously be formulated into various home or personal care or health care compositions. Such compositions will generally contain further ingredients to enhance performance and/or consumer acceptability.

Accordingly, other ingredients typically found in home or personal care or health care compositions may be added to the aqueous gel according to the invention.

Notably, the aqueous gels of the invention are tolerant to relatively high levels of C₂₋₄ monohydric alcohols (such as ethanol) and so have a particular applicability in the formulation of skin-cooling, skin-soothing or hand-sanitising preparations.

In such preparations, the amount of C₂₋₄ monohydric alcohol (such as ethanol) will generally range from 2 to 50%, preferably from 5 to 40% (by total weight C₂₋₄ monohydric alcohol based on the total weight of the aqueous gel).

The aqueous gels of the invention also have a particular utility in pumpable or especially sprayable formats. Sprays are popular delivery systems due to their ease of use, but usually require very watery, runny formulations. The gels of the invention offer non-drip benefits as well as pleasant aesthetics and rheological properties. In particular, they can be formulated to have thixotropic (shear-thinning) properties so that they can pass through a spray nozzle, creating a fine mist, yet reform as a viscous gel on the target surface, such as the skin.

Accordingly, the aqueous gels of the invention may advantageously be formulated into pumpable or sprayable home or personal care or health care compositions, which are suitable for dispensing from a pump or spray dispensing package, such as a hand-actuated trigger spray package. Examples of such compositions include sun protection sprays, wet wipe concentrates (for spraying onto fabric), wound healing gels (such as spray-on plaster), joint or muscle rub preparations, after-shave cooling sprays and non-drip surface coatings.

The aqueous gels of the present invention also provide excellent tactile properties, in particular superior skin feel and reduced stickiness.

Particularly good results have been observed when formulating with active ingredients such as glucosamine, which is used in topical healthcare formulations.

Accordingly, a preferred aqueous gel according to the invention comprises glucosamine and/or one or more monomeric glucosamine derivatives such as D-glucosamine hydrochloride, D-glucosamine sulphate, D-glucosamine iodide or other salts of D-glucosamine; N-acetyl D-glucosamine and its salts; chitin hydrolysate, chitosan hydrolysate, glucosamine phosphates, sulfates, or acetates and their salts; D-glucosaminic acid and N-acetyl D-glucosamine phosphates, sulfates and their salts.

When present, the amount of glucosamine and/or monomeric glucosamine derivative in the aqueous gel of the invention generally ranges from 0.1 to 5wt %, and preferably ranges from 1 to 3wt % (by total weight glucosamine and/or monomeric derivative thereof based on the total weight of the aqueous gel).

Other active ingredients which may suitably be incorporated into aqueous gels of the invention include water-soluble film-forming resins suitable for imparting hold and style to hair. Good skin and hair feel and particularly reduced stickiness have been observed when formulating with these materials. The resin is preferably nonionic. Illustrative nonionic resins include polyvinylpyrrolidone (PVP), copolymers of PVP and methylmethacrylate, copolymers of PVP and vinyl acetate (VA), polyvinyl alcohol (PVA), copolymers of PVA and crotonic acid, copolymers of PVA and maleic anhydride, hydroxypropyl cellulose, hydroxypropyl guar gum, PVP/ethylmethacrylate/methacrylic acid terpolymer, vinyl acetate/crotonic acid/vinyl neodecanoate copolymer, octylacrylamide/acrylates copolymer, monoethyl ester of poly(methyl vinyl ether/maleic acid) and mixtures thereof.

When present, the amounts of these film-forming resins may range from 0.5 to 10%, preferably from 1 to 8%, optimally from 1.5 to 4% (by total weight film-forming resin based on the total weight of the aqueous gel).

The aqueous gels of the invention also provide sufficient structure for the suspension of a variety of particulate materials. Examples include solid organic or inorganic particulates such as wax beads, polymer beads, encapsulates (such as perfume encapsulates or vitamin encapsulates) and glitter or sparkle particles (such as mica flakes).

When present, the amounts of these particulate materials may suitably range from 0.1 to 3%, preferably from 0.5 to 2% (by total weight particulate material based on the total weight of the aqueous gel).

Other optional ingredients typically found in home or personal care or health care compositions may also be added to the aqueous gel according to the invention.

Such ingredients will generally be present individually in an amount ranging from 0 to 5% by weight individual ingredient based on the total weight of the aqueous gel.

Examples of such further optional ingredients include fragrances, water-soluble dyes, preservatives, trace elements, and other hydrophilic active elements such as hydrophilic sun filters, botanical extracts, bacterial extracts, proteins or their hydrolysates (e.g. elastin or collagen hydrolysates), and moisturizers.

The invention is further illustrated with reference to the following, non-limiting examples. All concentrations are expressed by weight percent of the total formulation, and as level of active matter.

### EXAMPLES

### Example 1

The following formulation represents an aqueous gel according to the invention.

| Ingredient | (wt %) |
|---|---|
| Oxidised cellulose⁽¹⁾ | 2 |
| Glycerine | 2 |
| Denatured ethanol | 10 |
| Immortelle extract | 2 |
| Curry leaf extract | 1 |
| Purified water | q.s. |

⁽¹⁾Partially and selectively oxidised cellulose as described in WO2010/076292

### Method of Manufacture

1) 80 wt % of an aqueous solution of the oxidised cellulose⁽¹⁾ (2.5 wt % a.i.) is added to a main vessel and the remaining water added with mechanical stirring
2) Once uniform the glycerine, denatured ethanol and the extracts are added individually with continuous stirring
3) Mixing is continued until uniform

A firm gel is obtained with a pH of 5.96 and a viscosity of about 23,200 mPa·s (Brookfield RVT Viscometer, Spindle 7, 2.5 rpm, measured after 30 seconds). It is suitable for use as a cooling and soothing moisture spray gel. It is able to break down when passing through a spray nozzle to produce a fine mist, yet fully reform on the skin surface as a gel.

### Example 2

The following formulation represents an aqueous gel according to the invention.

| Ingredient | (wt %) |
|---|---|
| Oxidised cellulose⁽¹⁾ | 2 |
| Glycerine | 2 |
| Botanical extract | 2 |
| Purified water | q.s. |

A firm gel is obtained with a pH of 6.01 and a viscosity of about 25,600 mPa·s (Brookfield RVT Viscometer, Spindle 7, 2.5 rpm, measured after 30 seconds). It is able to break down when passing through a spray nozzle to produce a fine mist, yet fully reform on the skin surface as a gel.

Comparing the properties of Example 1 and Example 2 respectively, it can be seen that the presence of ethanol in the former does not significantly affect stability or flow characteristics.

### Example 3

The following formulation represents an aqueous gel according to the invention.

| Ingredient | (wt %) |
|---|---|
| Oxidised cellulose⁽¹⁾ | 1.5 |
| Sodium chloride | 0.4 |
| Glycerine | 2 |
| Glucosamine sulphate | 2 |
| Methylisothiazolinone | 0.6 |
| Jojoba beads | 0.5 |
| Purified water | q.s. |

### Method of Manufacture

1) 60 wt % of an aqueous solution of the oxidised cellulose⁽¹⁾ (2.5 wt % a.i.) is added to a main vessel
2) The sodium chloride is dispersed in 50% of the remaining water and this is added to the main vessel with continuous stirring, mixing until uniform
3) The glucosamine is dispersed in the remaining 50% of water and this is added to the main vessel with continuous stirring
4) The methylisothiazolinone is dispersed in the glycerine and this is added to the main vessel with continuous stirring and mixed until completely uniform
5) The jojoba beads are added and dispersed with stirring

A gel is obtained with a pH of 6.17 and a viscosity of about 2,100 mPa·s (Brookfield RVT Viscometer, Spindle 7, 2.5 rpm, measured after 30 seconds). The gel is low viscosity but nevertheless provides sufficient structure for stable suspension of the dense wax beads. The gel is also non-drip and non-sticky with a pleasant skin feel and is suitable for use as a topical muscle/joint care preparation.

### Example 4

The following formulation represents an aqueous gel according to the invention.

| Ingredient | (wt %) |
|---|---|
| Oxidised cellulose⁽¹⁾ | 1.5 |
| Ethanol | 35 |
| Methylisothiazolinone | 0.6 |
| Purified water | q.s. |

A gel is obtained with a pH of 5.84 and a viscosity of about 4,200 mPa·s (Brookfield RVT Viscometer, Spindle 7, 2.5 rpm, measured after 30 seconds). The gel has a pleasant skin feel with superior tactile properties and less drying of the skin with continued use. It is suitable as an alcoholic hand sanitizer.

### Example 5

The following formulation represents an aqueous gel according to the invention.

| Ingredient | (wt %) |
|---|---|
| Oxidised cellulose⁽¹⁾ | 1.5 |
| Sodium chloride | 0.5 |
| Glucosamine sulphate | 2 |
| Ethanol | 6.2 |
| Methylisothiazolinone | 0.6 |
| Purified water | q.s. |

A gel is obtained with a pH of 6.18 and a viscosity of about 2,000 mPa·s (Brookfield RVT Viscometer, Spindle 7, 2.5 rpm, measured after 30 seconds). The gel has a pleasant skin feel with reduced skin stickiness and the ability to apply the product and allow it to dry without needing to then wash hands. It is suitable as a topical glucosamine gel.

1. An aqueous comprising:
a) from 0.5 to 5 wt % dispersed modified cellulose biopolymer, wherein the modification consists of the cellulose having its C6 primary alcohols oxidised to carboxyl moieties (acid/COOH—) on 10 to 70% of the glucose units and substantially all the remainder of the C6 positions occupied by unmodified primary alcohols;
a water-soluble or water-miscible organic non-solvent for the modified cellulose biopolymer;
0 to 10 wt % non-surfactant electrolyte, and water;
in which the aqueous gel comprises less than 3 wt % oil phase ingredients.

2. An aqueous gel according to claim 1, in which the amount of the modified cellulose biopolymer ranges from 1 to 2 wt % (by total weight modified cellulose biopolymer based on the total weight of the aqueous gel).

3. An aqueous gel according to claim 1, in which the water-soluble or water-miscible organic non-solvent is a mono- or polyhydric alcohol.

4. An aqueous gel according to claim 1, in which the total content of anionic surfactant and amphoteric surfactant and cationic surfactant and nonionic surfactant amounts to less than 0.1% by weight based on the total weight of the aqueous get

5. An aqueous gel according to, claim 1, which comprises from 2 to 50 wt % C₂₋₄ monohydric alcohol.

6. An aqueous gel according to claim 1 which comprises from 0.1 to 5 wt % glucosamine and/or monomeric glucosamine derivative.

7. An aqueous gel according to claim 1 which has a viscosity ranging from 1,500 to 30,000 mPa·s (Brookfield RVT Viscometer, Spindle 7, 2.5 rpm, measured after 30 seconds).

8. An aqueous gel according to claim 1 which is packaged in a pump or spray dispenser.
